(19) United States
(12) Patent Application Publication
(10) Pub. No.: US 2016/0121006 A1
(43) Pub. Date: May 5, 2016

(54) HANDHELD ELECTRONIC DEVICE AND ANTIBACTERIAL METHOD OF THE SAME

(71) Applicant: HTC Corporation, Taoyuan City (TW)

(72) Inventor: Chun-I Wu, Taoyuan City (TW)

(21) Appl. No.: 14/528,602

(22) Filed: Oct. 30, 2014

(51) Int. Cl. A61L 2/10; G06F 3/00; G09G 3/32

(52) U.S. Cl. CPC A61L 2/10; G09G 3/3208; G06F 3/002; G09G 2360/144; G09G 2360/141; G09G 2320/08; G09G 2354/00

(57) ABSTRACT

A handheld electronic device having an antibacterial mechanism is provided. The handheld electronic device includes a main body, a display panel and a processing module. The display panel is disposed at a first surface of the main body and includes a plurality of display light sources and at least one antibacterial light source. The display light sources generate a display light. The antibacterial light source generates an antibacterial light. The processing module is coupled to the display light sources and the antibacterial light source. The processing module is operated to activate the display light sources to generate the display light during a display period and to activate the antibacterial light source to generate the antibacterial light during an antibacterial period.

### BACKGROUND

1. Field of Invention

The present invention relates to a handheld electronic device. More particularly, the present invention relates to a handheld electronic device and an antibacterial method of the same.

2. Description of Related Art

Handheld electronic devices such as smartphones and tablet PCs become the most important electronic products due to their light weight, powerful data-processing and data-transmission ability and integration of various multimedia technologies.

However, since the handheld electronic device is hold by the user for a long time during operation or is often disposed close to the user's face to perform the phone-call communication, the bacteria grows thereon. According to some experimental statistics, there are 2700 to 4200 units of Escherichia coli on the handheld electronic device, where 100 milliliter (ml) of drinking water only includes no more than one unit of Escherichia coli. If there is no appropriate antibacterial mechanism, the user may be infected by the bacteria to cause the health problem.

Accordingly, what is needed is a handheld electronic device and antibacterial method of the same to address the above issues.

### SUMMARY

An aspect of the invention is to provide a handheld electronic device having an antibacterial mechanism. The handheld electronic device includes a main body, a display panel and a processing module. The display panel is disposed at a first surface of the main body and includes a plurality of display light sources and at least one antibacterial light source. The display light sources are configured to generate a display light for generating an image. The antibacterial light source is configured to generate an antibacterial light. The processing module is coupled to the display light sources and the antibacterial light source. The processing module is operated to activate the display light sources to generate the display light during a display period and to activate the antibacterial light source to generate the antibacterial light during an antibacterial period.

Another aspect of the present invention is to provide an antibacterial method used in a handheld electronic device that includes a main body, a display panel and a processing module, wherein the display panel is disposed at a first surface of the main body and includes a plurality of display light sources and at least one antibacterial light source and the processing module is disposed in the main body. The antibacterial method includes the steps outlined below. The handheld electronic device is operated in a display period to activate the display light sources to generate a display light for generating an image. Whether the display panel is under an unused state is determined. The handheld electronic device is operated in an antibacterial period to kill bacteria on the first surface by activating the antibacterial light source to generate the antibacterial light.

These and other features, aspects, and advantages of the present invention will become better understood with reference to the following description and appended claims.

It is to be understood that both the foregoing general description and the following detailed description are by examples, and are intended to provide further explanation of the invention as claimed.

### BRIEF DESCRIPTION OF THE DRAWINGS

The invention may be more fully understood by reading the following detailed description of the embodiment, with reference made to the accompanying drawings as follows:

FIG. 1A is a block diagram of a handheld electronic device in an embodiment of the present invention;

FIG. 1B is a perspective view of the handheld electronic device in FIG. 1A in an embodiment of the present invention;

FIG. 1C is a cross-sectional view of the display panel in FIG. 1A in an embodiment of the present invention;

FIG. 2 is a cross-sectional view of the display panel in FIG. 1A in an embodiment of the present invention;

FIG. 3 is a partial cross-sectional view of the display panel in FIG. 1A in another embodiment of the present invention;

FIG. 4 is a block diagram of a handheld electronic device in an embodiment of the present invention;

FIG. 5 is a three dimensional diagram of the handheld electronic device illustrated in FIG. 4 in an embodiment of the present invention;

FIG. 6 is a cross-sectional view of the handheld electronic device in FIG. 4 in another embodiment of the present invention; and

FIG. 7 is a flow chart of an antibacterial method used in the handheld electronic device in an embodiment of the present invention.

### DETAILED DESCRIPTION

Reference will now be made in detail to the present embodiments of the invention, examples of which are illustrated in the accompanying drawings. Wherever possible, the same reference numbers are used in the drawings and the description to refer to the same or like parts.

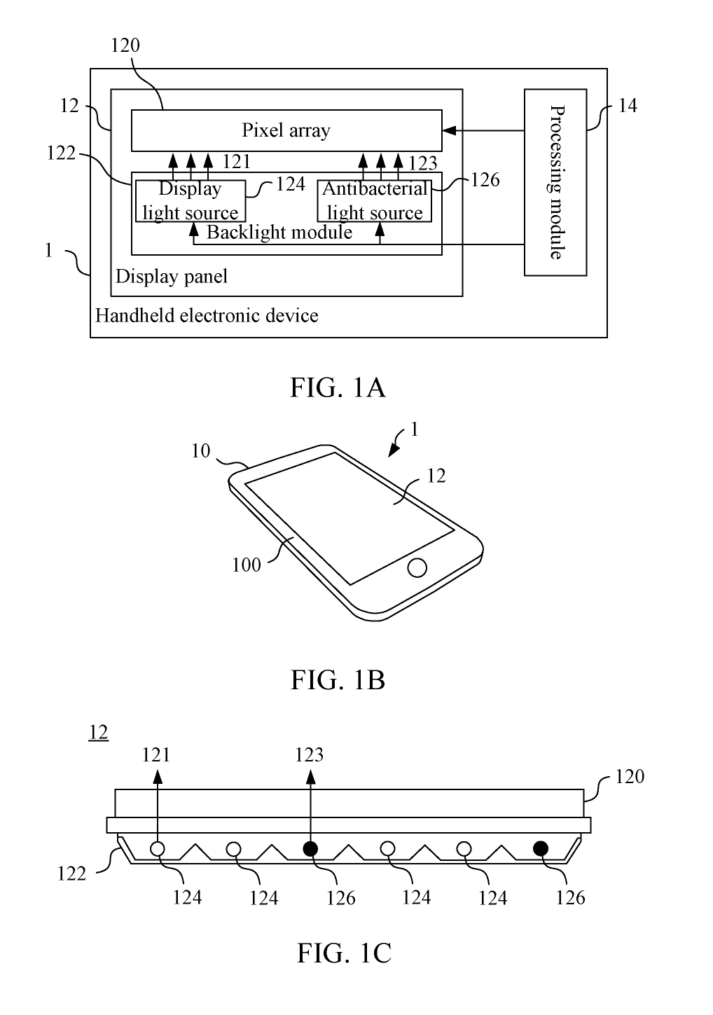

Reference is now made to FIG. 1A to FIG. 1C. FIG. 1A is a block diagram of a handheld electronic device 1 in an embodiment of the present invention. The handheld electronic device 1 includes a main body 10, a display panel 12 and a processing module 14. FIG. 1B is a perspective view of the handheld electronic device 1 in FIG. 1A in an embodiment of the present invention. FIG. 1C is a cross-sectional view of the display panel 12 in FIG. 1A in an embodiment of the present invention.

The display panel 12 is correspondingly disposed at a first surface 100 of the main body 10. In the present embodiment, the display panel 12 includes a pixel array 120 and a backlight module 122. The display panel 12 may be such as, but not limited to a thin film transistor (TFT) liquid crystal display panel. Therefore, the pixel array 120 may include a plurality of thin film transistors (not illustrated). In different embodiments, the pixel array 120 may further selectively include such as, but not limited to a substrate for supporting the thin film transistors, liquid crystal molecules, spacers, a polyimide alignment film, a light filter, a polarizer, etc (not illustrated) to accomplish the display mechanism.

In the present embodiment, the backlight module 122 is a bottom lighting backlight module disposed correspondingly under the pixel array 120. The backlight module 122 includes display light sources 124 and an antibacterial light sources 126. In FIG. 1A, one display light source 124 and one antibacterial light source 126 are exemplarily depicted. The display light source 124 is configured to generate a display light 121 for generating a display image. The antibacterial light sources 126 are configured to generate an antibacterial light 123 that may be used to kill bacteria. In an embodiment, the antibacterial light 123 is an ultraviolet light.

The actual number and the distribution relation between the display light source 124 and the antibacterial light sources 126 may be different according to the practical application. For example, in the example illustrated in FIG. 1C, each of the antibacterial light sources 126 may be disposed between every two display light sources 124. In other embodiments, other numbers and distribution relations may be used and are not limited by those illustrated in FIG. 1C.

In an embodiment, the backlight module 122 may selectively include a reflective plate 128 to make the display light 121 and the antibacterial light 123 intensively emerge toward the direction of the pixel array 120.

The processing module 14 is coupled to the display light sources 124 and the antibacterial light sources 126. The processing module 14 is configured to be operated to activate the display light sources 124 to generate the display light 121 during a display period and to activate the antibacterial light sources 126 to generate the antibacterial light 123 to kill bacteria on the first surface 100 during an antibacterial period.

In an embodiment, the antibacterial light sources 126 are not activated to avoid doing harm to the user. Further, since the antibacterial period is operated in an unused state, in which the user does not use the handheld electronic device 1 under the unused state, the display light sources 124 are not activated during the antibacterial period.

In an embodiment, the processing module 14 is configured to determine that the display panel 12 is under the unused state when the display panel 12 is in a standby mode to operate the handheld electronic device 1 in the antibacterial period. On the contrary, the processing module 14 is configured to determine that the display panel 12 is under the used state when the display panel 12 is in operation to operate the handheld electronic device 1 in the display period. In an embodiment, the operation state of the display panel 12 may be determined according to such as, but not limited to a power-supplying condition thereof.

As a result, the handheld electronic device 1 may use the antibacterial light sources 126 different from the display light sources 124 to generate the antibacterial light 123 to kill bacteria on the display panel 12. The condition of the growth of bacteria on the surface of the handheld electronic device 1 that may cause the infection of the user may be avoided.

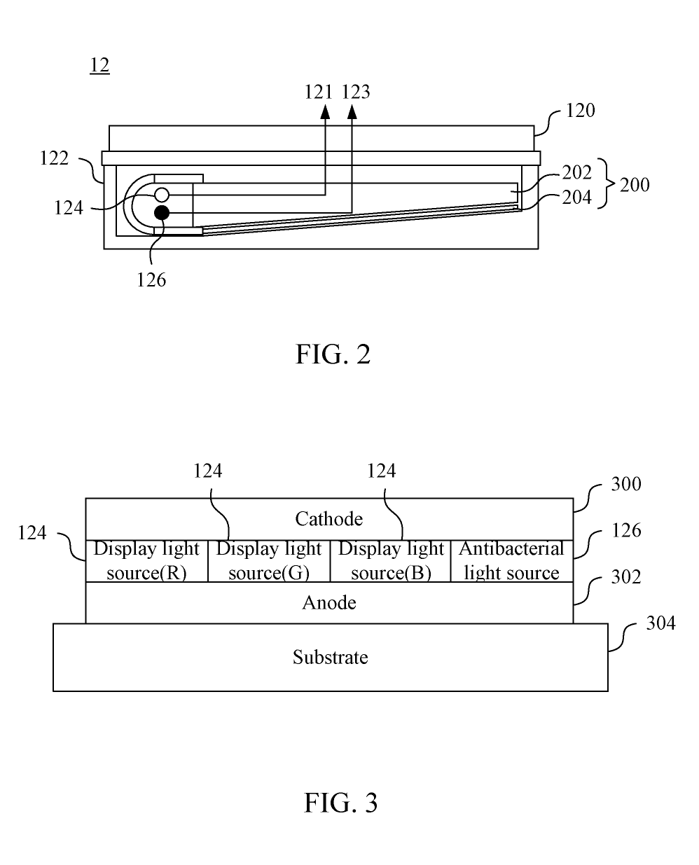

Reference is now made to FIG. 2. FIG. 2 is a cross-sectional view of the display panel 12 in FIG. 1A in an embodiment of the present invention.

In the present embodiment, the backlight module 122 included in the display panel 12 is an edge lighting backlight module disposed at at least one side of the pixel array 120. The display panel 12 further includes a light-guiding module 200 that includes such as, but not limited to a light-guiding plate 202 and a reflective plate 204 correspondingly disposed under the pixel array 120. The light-guiding module 200 is configured to guide the display light 121 generated by the display light sources 124 and the antibacterial light 123 generated by the antibacterial light sources 126 to the pixel array 120.

Reference is now made to FIG. 3. FIG. 3 is a partial cross-sectional view of the display panel 12 in FIG. 1A in another embodiment of the present invention. In FIG. 3, only a single pixel of the display panel 12 is illustrated.

In the present embodiment, the display panel 12 is an organic light-emitting diode (OLED) display panel. The organic light-emitting diode of each pixel included in the display panel 12 is formed between the cathode 300 and the anode 302 and is supported by the substrate 304. In other embodiments, the display panel 12 may selectively include an electron injection layer and an electron transport layer (not illustrated) that are electrically connected between the cathode 300 and the organic light-emitting diode, and a hole injection layer and a hole transport layer (not illustrated) that are electrically connected between the anode 302 and the organic light-emitting diode.

In a pixel, the display light source 124 is the red, green and blue organic light-emitting diodes included therein, which are labeled as R, G and B in FIG. 3. Moreover, the antibacterial light sources 126 may be such as, but not limited to an ultraviolet organic light-emitting diode correspondingly disposed therein. However, it is noted that in different embodiments, the antibacterial light sources 126 may be disposed in each pixel of the display panel 12 or be disposed in every certain number of pixels of the display panel 12, which is not limited thereto.

Accordingly, the handheld electronic device 1 of the present invention may be implemented in different types of display panels 12, such as but not limited to the thin film transistor liquid crystal display panel equipped with the bottom lighting backlight module and the edge lighting backlight module, and the organic light-emitting diode display panel in the embodiments described above. It is noted that the thin film transistor liquid crystal display panel equipped with the bottom lighting backlight module and the edge lighting backlight module and the organic light-emitting diode display panel are merely an example. In other embodiments, other structures of display panel may possibly be used. The structure of the display panel is not limited to those illustrated in FIG. 1C, FIG. 2 and FIG. 3.

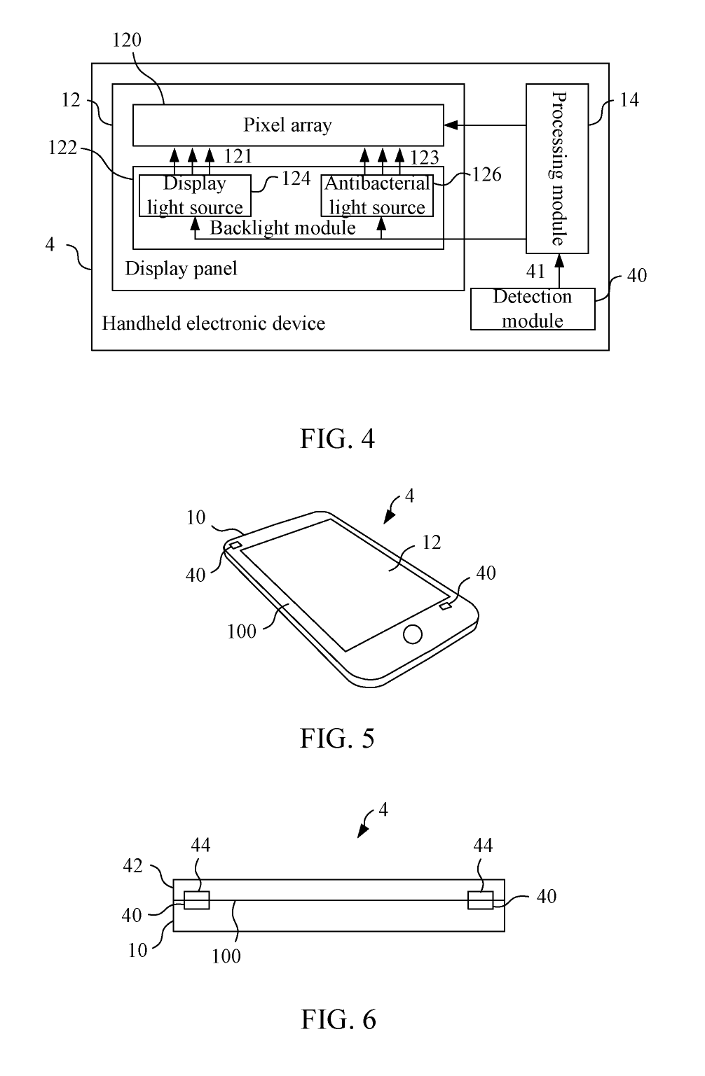

Reference is now made to FIG. 4 and FIG. 5. FIG. 4 is a block diagram of a handheld electronic device 4 in an embodiment of the present invention. FIG. 5 is a three dimensional diagram of the handheld electronic device 4 illustrated in FIG. 4 in an embodiment of the present invention,

Similar to the handheld electronic device 1 illustrated in FIG. 1A to FIG. 1C, the handheld electronic device 4 includes the main body 10, the display panel 12 and the processing module 14. The structure and the function of these components are identical to those in FIG. 1 and are not described in detail herein. In the present embodiment, the handheld electronic device 4 includes a detection module 40.

The detection module 40 is configured to detect an environment sensing value 41 related to the first surface 100 such that the processing module 14 determines that whether the display panel 12 is under the unused state to operate the handheld electronic device 4 in the antibacterial period when the display panel 12 is under the unused state.

In an embodiment, the detection module 40 includes a light sensor and the environment sensing value 41 is an environment light sensing value. The light sensor is disposed on the first surface 100 and is configured to detect an environment light on the first surface 100 to generate the environment light sensing value. The processing module 14 is configured to determine that the display panel 12 is under the unused state when the environment light sensing value is smaller than a specific value.

For example, when the handheld electronic device 4 is disposed on such as, but not limited to a surface of a desk such that the first surface 100 of the handheld electronic device 4 is blocked, the environment light sensing value generated according to the environment light is smaller than the specific value. The processing module 14 thus determines that he display panel 12 is under the unused state.

In another embodiment, the detection module 40 includes a touch sensor, and the environment sensing value 41 is a touch sensing value. The touch sensor is disposed on the first surface 100 and is configured to detect a touch action on the first surface 100 to generate the touch sensing value. In an embodiment, the touch sensing value generated by the touch sensor is a pressure value generated due to the touch action applied on the first surface 100. The processing module 14 is configured to determine that the display panel is under the unused state when the touch sensing value is larger than a specific value.

For example, when the handheld electronic device 4 is disposed on such as, but not limited to a surface of a desk such that a pressure is generated on the first surface 100 of the handheld electronic device 4 due to the gravity, the touch sensing value generated according to the touch action is larger than the specific value. The processing module 14 thus determines that the display panel 12 is under the unused state.

In yet another embodiment, the detection module 40 a motion sensor, and the environment sensing value 41 is a displacement sensing value. The motion sensor is disposed in the main body 10 and is configured to detect a motion of the main body 10 to generate the displacement sensing value. The processing module 14 is configured to determine that the display panel 12 is under the unused state when the displacement sensing value is smaller than a specific value.

For example, when the handheld electronic device 4 is disposed on such as, but not limited to a surface of a desk such that the handheld electronic device 4 has no displacement, the displacement sensing value generated according to the motion of the handheld electronic device 4 is smaller than the specific value. The processing module 14 thus determines that the display panel 12 is under the unused state.

Reference is now made to FIG. 4 and FIG. 6 at the same time, in which FIG. 6 is a cross-sectional view of the handheld electronic device 4 in FIG. 4 in another embodiment of the present invention. In the present embodiment, the handheld electronic device 4 further includes a back cover 42. Different designs of the back cover 42 may be applied in different embodiments to cover the first surface 100.

In the present embodiment, the back cover 42 includes a magnetic element 44, wherein the detection module 40 includes a hall sensor. The hall sensor is disposed in the main body 10 and is configured to detect a distance between the magnetic element 44 and the hall sensor to generate a distance sensing value. The processing module 14 is configured to determine that the first surface 100 is covered by the back cover 42 and the display panel 12 is under the unused state when the distance sensing value is smaller than a specific value.

For example, when the back cover 42 stays close or deviate from the first surface 100, the hall sensor senses the change of the amount of magnetic flux to determine the distance therebetween. When the back cover 42 covers the first surface 100, the distance sensing value generated according to the amount of the magnetic flux detected by the detection module 40 is smaller than the specific value. The processing module 14 thus determines that the display panel 12 is under the unused state.

It is noted that the detection module 40 implemented by using the light sensor or the touch sensor may also be used to detect the back cover 42 in the present embodiment. When environment light sensing value is smaller than a specific value due to the coverage of the back cover 42 or when the touch sensing value is larger than a specific value due to the touch action, the processing module 14 determines that the display panel is under the unused state.

In an embodiment, the detection module 40 may selectively include at least two kinds of sensors mentioned above. The processing module 14 may determine that whether the display panel 12 is in the unused state according to the sensing value of each of the sensors together with the standby mode of the display panel 12 to accomplish a more accurate determination result.

It is noted that the mechanism of determining that whether the display panel 12 is in the unused state may be performed in every fixed time interval set by the processing module 14. In an embodiment, the processing module 14 may further perform the determining mechanism using the sensing values of sensors after the display panel 12 is determined to be in the standby mode. Moreover, in the above embodiments, two detection modules 40 are illustrated as an example. In other embodiments, the number of the detection modules 40 may be adjusted according to the practical condition and is not limited thereto.

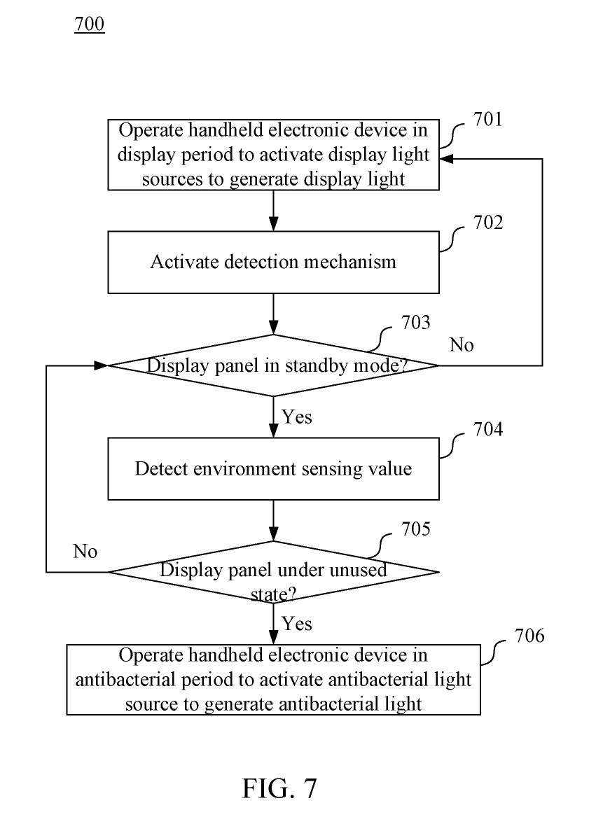

Reference is now made to FIG, 7. FIG. 7 is a flow chart of an antibacterial method 700 used in the handheld electronic device 1 in an embodiment of the present invention. The antibacterial method 700 may be used in the handheld electronic device 1 illustrated in FIG. 1A and FIG. 1B and the handheld electronic device 4 illustrated in FIG. 4 and FIG. 6. The antibacterial method 700 includes the steps outlined below (The steps are not recited in the sequence in which the steps are performed. That is, unless the sequence of the steps is expressly indicated, the sequence of the steps is interchangeable, and all or part of the steps may be simultaneously, partially simultaneously, or sequentially performed).

In step 701, the processing module 14 operates the handheld electronic device 1 in the display period to activate the display light sources 124 to generate the display light 121.

In step 702, the detection mechanism is activated.

In step 703, whether the display panel 12 is in a standby mode is determined. When the display panel 12 is not in the standby mode, the flow goes back to step 701 to keep operating the handheld electronic device 1 in the display period.

When the display panel 12 is in the standby mode, the detection module 40 further detects the environment sensing value in step 704.

In step 705, whether the display panel 12 is under the unused state is determined according to the environment sensing value. When the display panel 12 is not under the unused state, the flow goes back to step 703 to keep determining.

When the display panel 12 is under the unused state, the handheld electronic device 1 is operated in the antibacterial period in step 706 to activate the antibacterial light sources 126 to generate the antibacterial light 123 to kill bacteria on the first surface 100.

Although the present invention has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations may be made to the structure of the present invention without departing from the scope or spirit of the invention. In view of the foregoing, it is intended that the present invention cover modifications and variations of this invention provided they fall within the scope of the following claims.

What is claimed is:

1. A handheld electronic device, comprising:
a main body;
a display panel disposed at a first surface of the main body and comprising a plurality of display light sources and at least one antibacterial light source, wherein the display light sources are configured to generate a display light for generating an image and the antibacterial light source is configured to generate an antibacterial light; and
a processing module disposed in the main body, coupled to the display light sources and the antibacterial light source and is configured to be operated to activate the display light sources to generate the display light during a display period and to activate the antibacterial light source to generate the antibacterial light during an antibacterial period to kill bacteria on the first surface.

2. The handheld electronic device of claim 1, wherein the processing module is further configured to determine that the display panel is under an unused state when the display panel is in a standby mode to operate the handheld electronic device in the antibacterial period.

3. The handheld electronic device of claim 1, further comprising a detection module configured to detect an environment sensing value related to the first surface such that the processing module determines that whether the display panel is under an unused state to operate the handheld electronic device in the antibacterial period when the display panel is under the unused state.

4. The handheld electronic device of claim 3, wherein the detection module comprises a light sensor disposed on the first surface of the main body and configured to detect an environment light on the first surface to generate an environment light sensing value such that the processing module is configured to determine that the display panel is under the unused state when the environment light sensing value is smaller than a specific value.

5. The handheld electronic device of claim 3, wherein the detection module comprises a touch sensor disposed on the first surface of the main body and configured to detect a touch action on the first surface to generate a touch sensing value such that the processing module is configured to determine that the display panel is under the unused state when the touch sensing value is larger than a specific value.

6. The handheld electronic device of claim 3, further comprising a back cover that comprises a magnetic element, wherein the detection module comprises a hall sensor disposed in the main body and configured to detect a distance between the magnetic element and the hall sensor to generate a distance sensing value such that the processing module is configured to determine that the first surface is covered by the back cover and the display panel is under the unused state when the distance sensing value is smaller than a specific value.

7. The handheld electronic device of claim 3, wherein the detection module comprises a motion sensor disposed in the main body and configured to detect a motion of the main body to generate a displacement sensing value such that the processing module is configured to determine that the display panel is under the unused state when the displacement sensing value is smaller than a specific value.

8. The handheld electronic device of claim 1, wherein the display panel comprises:
a pixel array; and
a backlight module, wherein the display light sources and the antibacterial light source are comprised in the backlight module.

9. The handheld electronic device of claim 8, wherein the backlight module is a bottom lighting backlight module disposed correspondingly under the pixel array.

10. The handheld electronic device of claim 8, wherein the backlight module is an edge lighting backlight module disposed at at least one side of the pixel array, wherein the display panel comprises a light-guiding module disposed correspondingly under the pixel array and configured to guide the display light generated by the display light sources and the antibacterial light generated by the antibacterial light source to the pixel array.

11. The handheld electronic device of claim 1, wherein each of the display light sources and the antibacterial light source is an organic light-emitting diode (OLED) comprised in the display panel.

12. The handheld electronic device of claim 1, wherein the antibacterial light source is disposed in every certain number of the display light sources.

13. The handheld electronic device of claim 1 wherein the antibacterial light is an ultraviolet light.

14. An antibacterial method used in a handheld electronic device that comprises a main body, a display panel and a processing module, wherein the display panel is disposed at a first surface of the main body and comprises a plurality of display light sources and at least one antibacterial light source and the processing module is disposed in the main body, the antibacterial method comprises:
operating the handheld electronic device in a display period to activate the display light sources to generate a display light for generating an image;
determining that whether the display panel is under an unused slate;
operating the handheld electronic device in an antibacterial period to kill bacteria on the first surface by activating the antibacterial light source to generate the antibacterial light.

15. The antibacterial method of claim 14, further comprising determining that the display panel is under an unused state when the display panel is in a standby mode.

16. The antibacterial method of claim 14, wherein the handheld electronic device further comprises a detection module, the antibacterial method further comprises:
detecting an environment sensing value related to the first surface;
determining that whether the display panel is under an unused state according to the environment sensing value.

17. The antibacterial method of claim 16, wherein the detection module comprises a light sensor disposed on the first surface of the main body and configured to detect an environment light on the first surface to generate an environment light sensing value, the antibacterial method further comprises:
determining that the display panel is under the unused state when the environment light sensing value is smaller than a specific value.

18. The antibacterial method of claim 16, wherein the detection module comprises a touch sensor disposed on the first surface of the main body and configured to detect a touch action on the first surface to generate a touch sensing value, the antibacterial method further comprises;
determining that the display panel is under the unused state when the touch sensing value is larger than a specific value.

19. The antibacterial method of claim 16, wherein the handheld electronic device further comprises a back cover that comprises a magnetic element, wherein the detection module comprises a hall sensor disposed in the main body and configured to detect a distance between the magnetic element and the hall sensor to generate a distance sensing value, the antibacterial method further comprises:
determining that the first surface is covered by the back cover and the display panel is under the unused state when the distance sensing value is smaller than a specific value.

20. The antibacterial method of claim 16, wherein the detection module comprises a motion sensor disposed in the main body and configured to detect a motion of the main body to generate a displacement sensing value, the antibacterial method further comprises:
determining that the display panel is under the unused state when the displacement sensing value is smaller than a specific value.
